(19) United States
(12) Patent Application Publication
(10) Pub. No.: US 2003/0106140 A1
(43) Pub. Date: Jun. 12, 2003

(54) Diving mask

(76) Inventor: Meroni Bruno, Lodi (IT)

Correspondence Address: NOTARO & MICHALOS P.C., Empire State Building, Suite 6902, 350 Fifth Avenue, New York, NY, 10118-6985, US

(21) Appl. No.: 10/315,572

(22) Filed: Dec. 10, 2002

(30) Foreign Application Priority Data
Dec. 12, 2001 (IT) MI2001A 002620
Dec. 12, 2001 (IT) MI2001A 002621
Jul. 19, 2002 (IT) MI2002A 001602

(51) Int. Cl. A61F 9/02

(52) U.S. Cl. 2/427

(57) ABSTRACT

This invention relates to an improved diving mask, wherein the mask body shows, in cross section, a substantially truncated-conic shape or anyway with diverging walls starting from the glass area towards the face-supporting edge and without undercuts. This allows different advantages, among which, above all, to considerably approach the lenses to the face so as to guarantee a visual angle wider than the usual masks. Moreover the inner air volume is decreased with consequent less problems caused by the water pressure, when one dives at a certain depth.

The walls show preferably two pieces having a different thickness, with a first piece near the glasses, having a greater and substantially uniform thickness which supports the face and which connects to a second piece, which is thinner and has a thickness decreasing up to the extreme edge, to guarantee the watertight, without the need to resort to edges, tongues or complex embodiments which would involve the need to resort to complex moulds.

This purpose is reached by a diving mask wherein the flat glasses are positioned sloping with the outer edges placed backwards.

By choosing in an appropriate way the glass angular position it is possible to obtain a view wherein the objects appear with the real sizes and therefore to have a correct perception of their distance.

This invention proposes an improved diving mask, wherein the mask body shows, in cross-section, a truncated-conic shape or anyway provided with diverging walls starting from the glass area towards the edge leaning on the face, and without undercuts.

More particularly in the mask according to the invention, the mask body and the lenses are made of the same transparent material, for example platinic silicone and similar materials, by differentiating the thickness in the various areas so that in the parties reserved to come into contact with the face, where the facial part has to show a good watertight, the thickness be thin so as to allow the mask to deform and perfectly follow the face profile, while along the lens perimeter and the frontal, the thickness will be greater so that the structure well resist the outer pressure without deforming.

This allows different advantages, among which above all to considerably approach the lenses to the face so to ensure a visual angle wider than the usual masks and to reduce the air inner volume, with consequent less problems caused by the water pressure when one dives at a certain depth. Furthermore, according to a preferred embodiment, the mask shows the glasses or lenses which are flat and are positioned sloping, with the outer edges backward, allowing thereby the view with the correct perception of the distances and the sizes also underwater.

At last the mask according to the invention can be easily produced without the need to resort to moulds which comprises several movable parts, with the consequent decrease in the installation costs and therefore in the product.

The diving masks at present known show a section lightly tightening in the central area, comprised between the glasses and the edge leaning on the face, and this involves the need, for the production, to resort to complex moulds comprising several movable parts.

Moreover some perts are required, made of plastic material, for mounting the glass lenses on the soft body which leans on the face.

From this configuration many other problems or restrictions arise for the users, since the masks at present known allow an almost reduced visual angle and furthermore the air volume inside the mask, although reduced, can cause problems due to the pressure increase, when one dives at a certain depth.

The known masks, furthermore, show a projecting tongue or edge aiming at improving the watertight and positioned near the mask side edge in the inner part and also this feature requires, for the moulding, complex and expensive moulds.

At last with the known masks, which are provided with glasses positioned on the same plane before the user's eyes, there are problems for those who practise underwater activities, owing to a non-correct perception of the distances and sizes underwater, due to the fact that owing to refraction phenomena the objects seen through the mask glass usually utilised for diving, appear enlarged in the water.

The problem of a correct view underwater has not anyway found, until today, such a simple and economically practical solution to be applied on the common diving masks with a moderate cost.

The present invention relates to this field, and proposes an improved diving mask with an extremely simple concept and which does not show the above described drawbacks.

In particular in the mask according to the invention, the body shows a section having a not-decreasing width starting from the lenses towards the outer edge, in particular a truncated-conic section, without undercuts, and this allows to reduce the mask inner volume and to realize the product with moulds simple and therefore more economic.

In an additional feature of the invention it is conceived to realize the mask body with a first piece, near the glasses, having a substantially constant greater thickness which connects to a second piece having a less thickness and which reduces up to the outer edge.

This allows to bring the lenses near the face so to enlarge in a considerable way the visual angle, to avoid that the mask deforms excessively when it is subject to the pressure, and to decrease thereby the volume of the inner air.

According to a feature of the invention, the lenses and the body are realised integral, in the same transparent material.

This allows, from the one hand, to use a sole mould for the production, enables to obtain a mask more flexible and more easily adaptable to the face shape, and allows to realise a less deep mask, with the consequence of a widening of the visual field (since the lens openings are closer to the eye) and less problems due to the pressure at high deepness.

In accordance with an advantageous feature of the invention, the mask is provided with “lenses” consisting of a pair of flat walls, made of glass or other plastic material, positioned sloping, with the outer edges backward. With this last solution it is possible to change the sizes of the image that the eye perceives, by adjusting the glass slope to obtain a correct perception of the distances and sizes.

This invention will be now described in detail, by way of a non-limitative example, with reference to the enclosed figures, wherein:

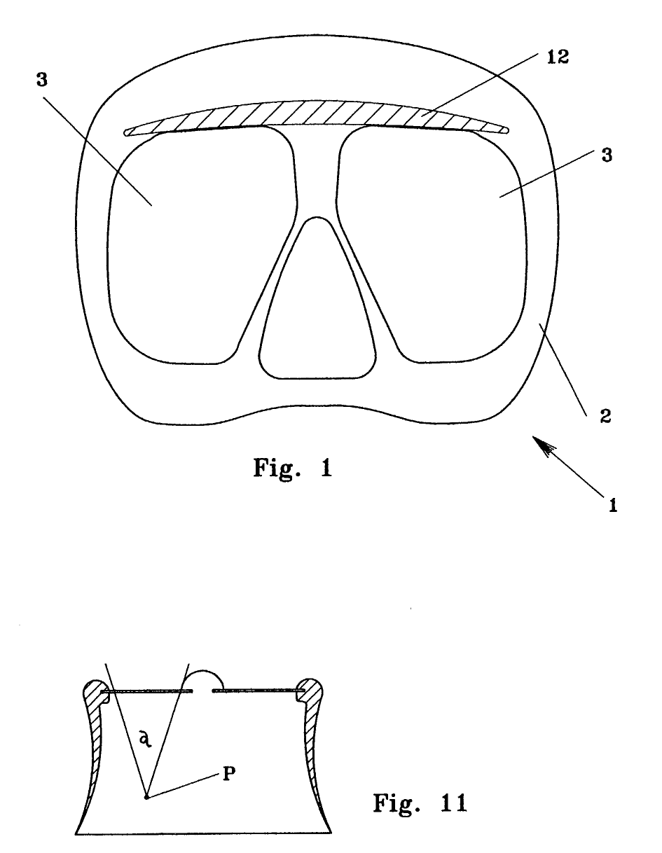

FIG. 1 is a front inner view of a mask according to the invention;

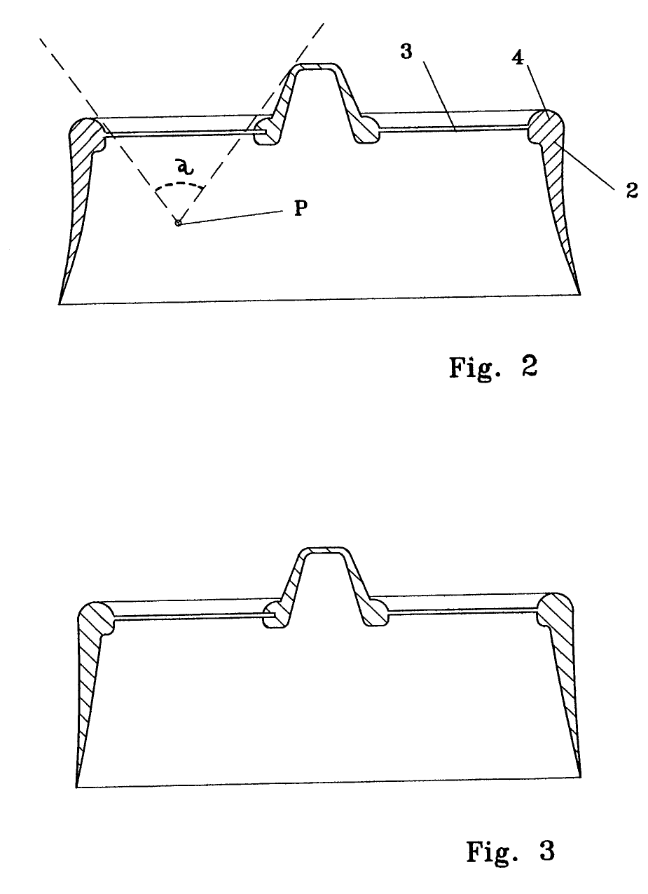

FIG. 2 is a cross-section of the mask according to the invention;

FIG. 3 show in cross-section another preferred embodiment of the mask according to the invention;

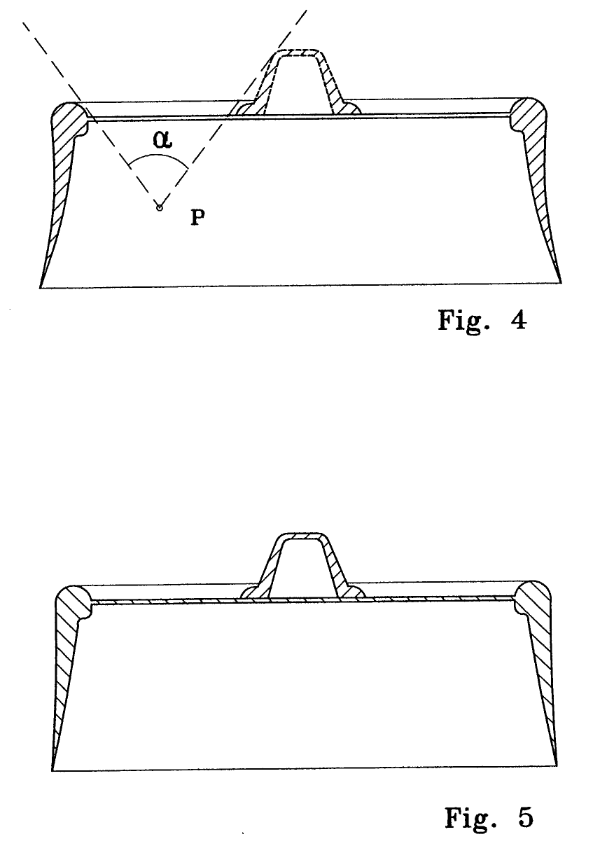

FIGS. 4 and 5 are cross-sections corresponding to those of FIG. 2 and 3 of an additional mask version with the front lens in an integral piece;

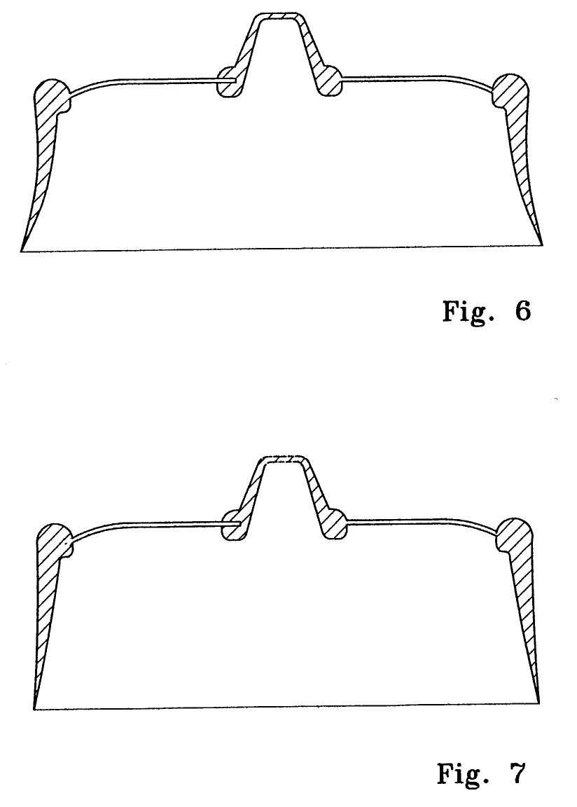

FIGS. 6 and 7 are cross-sections similar to those of FIGS. 2 and 3 of a mask, according to the invention, with curved lenses;

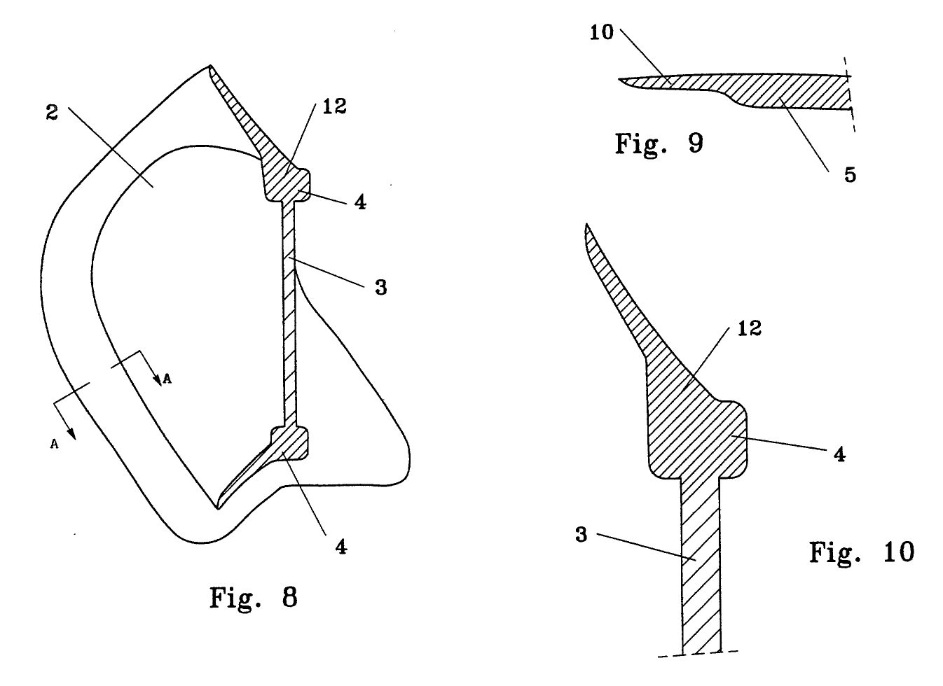

FIG. 8 is a vertical section of a mask according to the invention;

FIG. 9 shows in cross-section a detail of the mask of FIG. 8, seen in cross-section according to the line A-A;

FIG. 10 is an enlarged detail of the cross-section of the mask of FIG. 8;

FIG. 11 shows in cross-section a mask realised according to the known art;

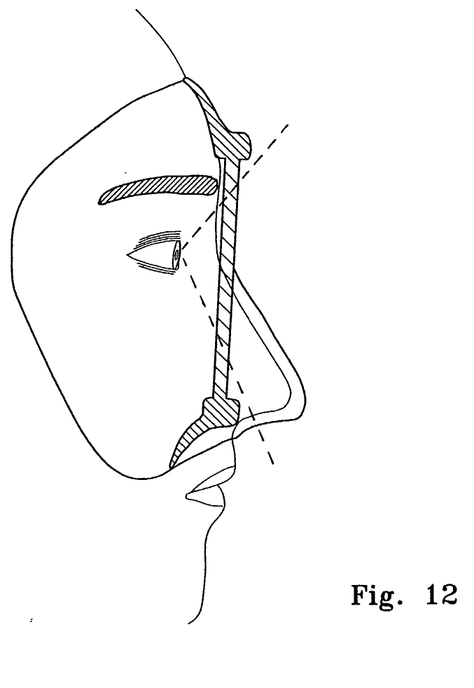

FIG. 12 shows the position of the mask on the face and underlines the particular closeness of the lenses to eyes;

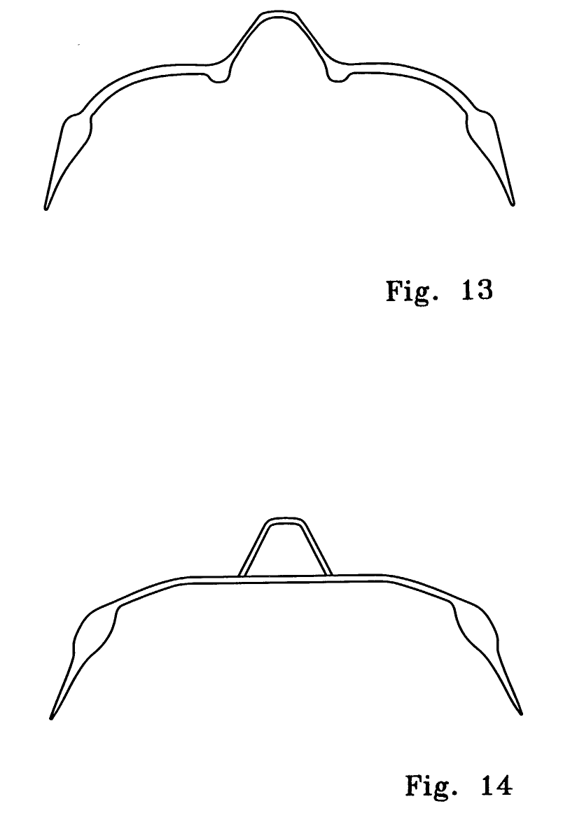

FIGS. 13 and 14 shows schematically, in horizontal section, an additional preferred version of the mask according to the invention, respectively with the lenses consisting of two separate glasses or elements in plastic material or a glass or element in plastic material in integral piece;

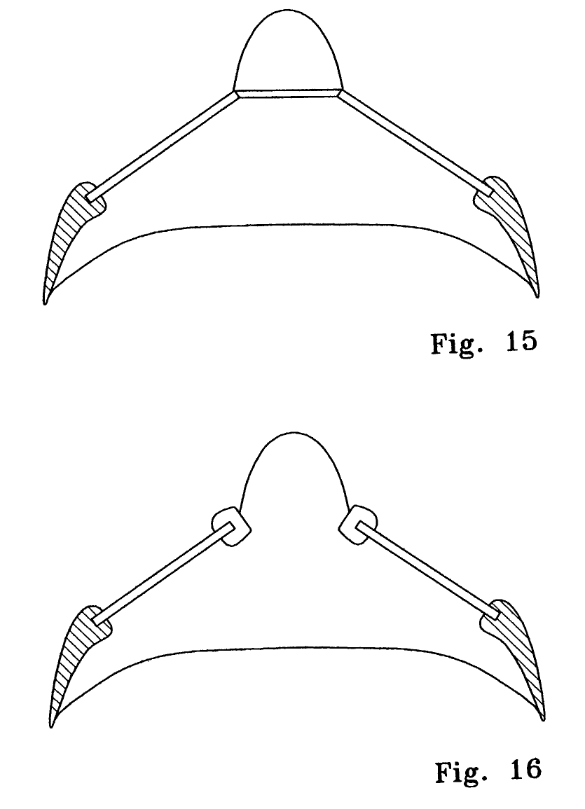

FIGS. 15 and 16 are cross-sections of an additional embodiment of the mask of the invention.

With reference to the enclosed figures, reference 1 indicates the mask according to the invention in its whole, which comprises a body 2 in flexible material, preferably of self-adhesive platinic silicone, to which a 1o pair of glasses or lenses made of glass or of plastic material, indicated with 3, are applied or co-moulded.

In the continuation and claims hereof, the word “frontal” will be used to indicate the mask portion wherein there are the glasses and the word “edge” to indicate the opposed end, which rests on the user's face.

The body 2 (FIG. 2) substantially consists of a wall which shows, from one side, a swelling 4, wherein the seat is drawn for the insertion of the glasses 3 and from the opposed side it thins out to rest on the user's face. According to the invention, the body 2 is shaped to have a cross-section which does not decreases starting from the frontal towards the edge, in particular a frusto-conical section as shown in FIG. 2 or, at most, a substantially cylindrical section, as shown in FIG. 3.

More particularly, the shape of the body 2 does not show undercuts, to enable the production of the mask by moulding or co-moulding by simply using a matrix and a punch without the need to resort to moulds complex and comprising movable parts.

In practice the central narrowing of the body 2, which is present on all the known masks, as highlighted in FIG. 11, is removed.

This solution allows to obtain remarkable advantages, in addition to the cheapness of the production due to the considerably lower cost of the apparatuses and the moulds used.

A first advantage is given by the increased visual angle with respect to the known masks.

The herein described embodiment allows, in fact, to realize a shorter mask, with the glasses nearer the face, so that the vertical and horizontal visual angle results much wider.

By way of example, FIGS. 2, 11 and 12 show this angle, indicated with “α”, while the letter P indicates which can be the position of the eye in the mask according to the invention, compared with the known masks.

Another advantage is given by the fact that the air volume contained in the is mask results to be lower than the known masks, with a considerable decrease in the problems which can take place further to the pressure increase, when one dives deeply.

Another advantage is a considerable improvement of the hydrodynamic resistance or friction, since the mask is very close to the face and has a very running profile (see FIGS. 2, 4 and 12).

In the mask according to the invention, the inner surface is free from undercuts and to guarantee an optimum watertight, the body wall is made to show, seen in cross-section, a first piece reference 5 and a second piece 10 (FIG. 9). The first piece 5 has a greater thickness, which thickness can be substantially uniform, and connects to a second piece 10, in correspondence with the edge, thinner and sloping towards the said edge.

This allows to obtain a mask showing such a sufficient surface resting on the face that it does not cause rubefaction also when the mask remains pressed for a lot of time against the face owing to the pressure, while the outer edge being thinner and sloping, perfectly fits to the face shape by securing the watertight, without the need to resort to edges, tongues and complex embodiments which should involve the need to resort to moulds, complex and expensive too.

This shape of the wall of the body 2 concerns all the mask, except for the area above the lenses where an enough thick edge, with reference 12 in the FIGS. 1, 8 and 10, shall be present, resting on the forehead and helping to keep the lenses in the correct position.

The lens area will be thinner and with a constant thickness, while the area around the lenses shall be swollen to realise an edge or bulb having a greater thickness to give the mask the necessary resistance not to deform excessively during the use.

The mask body thins out starting from the bulb 4 up to the extreme edge 10, where it reaches the minimum thickness.

In this way the part resting on the face results to be thin and flexible, the more it becomes soft the more it gets close to the edge, so that the pressure exerted by the water guarantees a perfect adherence of the mask to the face of the user.

The use of silicone, in particulaer self-adhesive platinic silicone for the realisation of the mask is particularly advantageous, since this material shows a deformability sufficient to allow a perfect adhesion of the mask to the face, it maintains the resilience features in the time and in addition thereto it can be transparent like glass, avoiding thereby to “close” completely the view.

Moreover it is antiallergic and can be moulded without treatments to lenses.

In a furteher ambodiment, the said lenses can be of glass or plastic, co-moulded together with the body, and can be positioned inside the mould through appropriate supports according to known technologies.

The silicone forming the mask body will form, all around the glass perimeter, swollen bulbs 4.

The transparency of the materials will permit a perfect view through the visibility areas as in the case of the traditional lenses made of glass or of rigid plastic material, in a mask that, contrary to the known masks, shows considerable advantages and allows a wider planning freedom of the face complex.

Furthermore the lenses can be provided, not only flat, but also curved or with at least a curved surface to allow optical corrections.

The mask according to the invention can have also different embodiments according to the type of glasses used.

In this way the same idea of solution can be applied to masks with the body having a diverging section or a straight wall with two flat glasses (FIGS. 2 and 3) or with a single flat glass (FIGS. 4 and 5) or with two curved glasses (FIGS. 6 and 7) or a single curved glass.

In particular according to a preferred embodiment of the invention shown in FIGS. 15 and 16, the lenses 3 are flat elements positioned in a sloping position, with the outer edges positioned backwards with respect to the inner edges, which are at the sides of the nose.

The optimum, slope of the lenses to see objects in their real sizes and obtain therefore, the exact perception of the distance can be determined experimentally.

The mask can have different sizes and, can be even realised in the form of swimming goggles, by simply avoiding the body portion 2 which covers the nose.

1) Improved diving mask characterized in that it provides for a mask body showing a section which does not reduce starting from the frontal to the edge resting on the face.

2) Improved diving mask according to claim 1, characterized in that the body shows a diverging or wedge-shaped form starting from the frontal to the edge, without undercuts.

3) A diving mask characterized in that it shows lenses realised integral with the facial part.

4) Improved diving mask according to claim 1, 2 or 3, characterized in that the inner surface of the mask is smooth and without tongues or undercuts.

5) Improved diving mask according to one of the preceding claims, characterized in that the body shows the wall divided into a first piece having a greater thickness, near the frontal, and which connects to a second piece, having a less thickness and decreasing to the edge resting on the face.

6) A diving mask according to claim 5, characterized in that the said first piece of greater thickness has a substantially constant thickness all over the mask wall, except for the area above the glasses, wherein the said body shows the wall shaped to realize an edge which leans on the forehead.

7) Improved diving mask according to one of the preceding claims, characterized in that it is made of platinic silicone.

8) Improved diving mask according to one of claims 1 to 6, characterized in that it is made of transparent, soft plastic material, in particular PVC.

9) Diving mask according to one of the preceding claims, characterized in that it provides for plane lenses in a sloping position with outer edges which are in a backward position with respect to the edges which are near the nose so as to obtain a view which provides for a correct perception of the distance and the sizes.

10) Diving mask according to one of the preceding claims, characterized in that the body and the leases are both realized by co-moulding techniques.

11) Diving mask according to the preceding claims, characterized in that it provides for a swollen edge all around the lens perimeter.

12) Diving mask according to the preceding claims, characterized in that the body is of self-adhesive platinic silicone co-moulded with the lenses.

13) Swimming goggle characterized in that it is realized by removing the nose covering portion from a mask according to one of the preceding claims.
